(19) United States
(12) Patent Application Publication
(10) Pub. No.: US 2009/0177047 A1
(43) Pub. Date: Jul. 9, 2009

(54) HEALTH WELLNESS MODELER

(75) Inventors: Pedro Martinez, Boca Raton, FL (US); Noel Guillama, Wellington, FL (US)

Correspondence Address: AKERMAN SENTERFITT, P.O. BOX 3188, WEST PALM BEACH, FL, 33402-3188, US

(73) Assignee: The Quantum Group, Inc., Wellington, FL (US)

(21) Appl. No.: 12/349,734

(22) Filed: Jan. 7, 2009

Related U.S. Application Data
(60) Provisional application No. 61/019,517, filed on Jan. 7, 2008.

(51) Int. Cl. A61B 5/00; G06N 5/02; G06F 17/30; G06G 7/60

(52) U.S. Cl. 600/300; 706/52; 707/104.1; 703/11; 707/E17.044

(57) ABSTRACT

A system for modeling the wellness of an individual. The system can include one or more electronic data processing elements. Additionally, the system can include a population profile integrator configured to execute on the electronic data processing element for integrating pre-determined health indicators with statistically-valid health data derived from a sample population. The system also can include an individual health profile comparator configured to execute on the electronic data processing element for comparing the integrated health indicators and health data to individual-specific health data. The system further can include a wellness modeler configured to execute on the electronic data processing element for generating a wellness assessment of an individual based on the comparison of the integrated health indicators and health data to the individual-specific health data. Optionally, the system can also include a re-calculation and projection tool configured to execute on the electronic data processing element for projecting probabilistic health results based on simulated variations of values of the individual-specific health data.

### CROSS-REFERENCE TO RELATED APPLICATIONS

This application claims the benefit of U.S. Provisional Patent Application No. 61/019,517, which was filed Jan. 7, 2008, and which is incorporated herein in its entirety.

### FIELD OF THE INVENTION

The present invention is related to the field of data processing, and more particularly, to computer-based systems and methods for processing health-relevant data for developing health wellness programs.

### BACKGROUND OF THE INVENTION

Continuing advances in medical research have helped to identify a number of key health indicators. The indicators can include measurable indicators, such as an individual's weight, blood pressure, and cholesterol level. The indicators also can include index-based values such as an individual's body mass index. Categorical data, such as whether or not an individual is a cigarette smoker, also can be a relevant health indicator.

Although such indicators can be used as generalized health predictors in a broad statistical sense, there is yet a need for efficient and effective techniques to aggregate and synthesize such indicators with other health-relevant data in a manner that can lead to the development of individualized health wellness programs tailored to particular individuals. Moreover, there is a need for efficient and effective techniques to aggregate and synthesize established health indicators with other health-relevant statistical data to generate an assessment of an individual's overall wellness and simulate the effects on the individual's wellness stemming from changes in the individual's behavior or lifestyle.

### SUMMARY OF THE INVENTION

The present invention is directed to systems and methods for integrating reliable measurements, or indicators, of health with statistically-valid samples of trends within a target population and overlaying the integrated data with individual-specific data in a manner that provides an accurate profile or assessment of an individual's wellness. Another aspect of the invention is the integration of reliable health indicators and statistically-based trend data with individual-specific data so as to simulate changes in the individual's wellness likely to occur from changes in the individual's behavior or lifestyle.

One object of the invention is to provide personalized statistical and numerical analysis of an individual's wellness, a pre-defined measure of the individual's current and prospective health, by integrating health indicators and demographic trends with individual-specific health factors, wherein the demographic trends can be periodically updated and wellness indicators adjusted accordingly. Another object of the invention is to model measurable wellness enhancements for an individual in response to proactive behavior of the individual, such as engaging in a weight-loss program, undergoing a cholesterol lowering regime, or quitting smoking. Still another object of invention is to provide individuals and/or organizations (e.g., corporation) a mechanism for incentivizing, motivating, and rewarding wellness-enhancing behavior. Yet another object of the invention is to provide meta data that can be extracted from individual-specific wellness data.

One embodiment of the invention is a system for modeling the wellness of an individual. The system can comprise one or more electronic data processing elements and a population profile integrator configured to execute on the electronic data processing element for integrating pre-determined health indicators with statistically-valid health data derived from a sample population. The system also can include an individual health profile comparator configured to execute on the electronic data processing element for comparing the integrated health indicators and health data to individual-specific health data. The system can further include a wellness modeler configured to execute on the electronic data processing element for generating a wellness assessment of an individual based on the comparison of the integrated health indicators and health data to the individual-specific health data.

Another embodiment of the invention is a method for developing an individualized wellness program. The method can include mapping a plurality of pre-determined health indicators to a database of health-related information and overlaying the pre-determined health indicators and health-related information to current individual-specific health data. The method also can include determining, based on the overlaying step, whether at least one individual health factor of an individual indicates a potential health problem. According to the method, if at least one health factor indicates a potential health problem, the potential health problem can be identified.

### BRIEF DESCRIPTION OF THE DRAWINGS

There are shown in the drawings, embodiments which are presently preferred. It is expressly noted, however, that the invention is not limited to the precise arrangements and instrumentalities shown.

FIG. 1 is a schematic view of a system for modeling the wellness of an individual, according to one embodiment of the invention.

FIG. 2 is flowchart of exemplary steps in a method for developing an individualized wellness program, according yet another embodiment of the invention.

### DETAILED DESCRIPTION

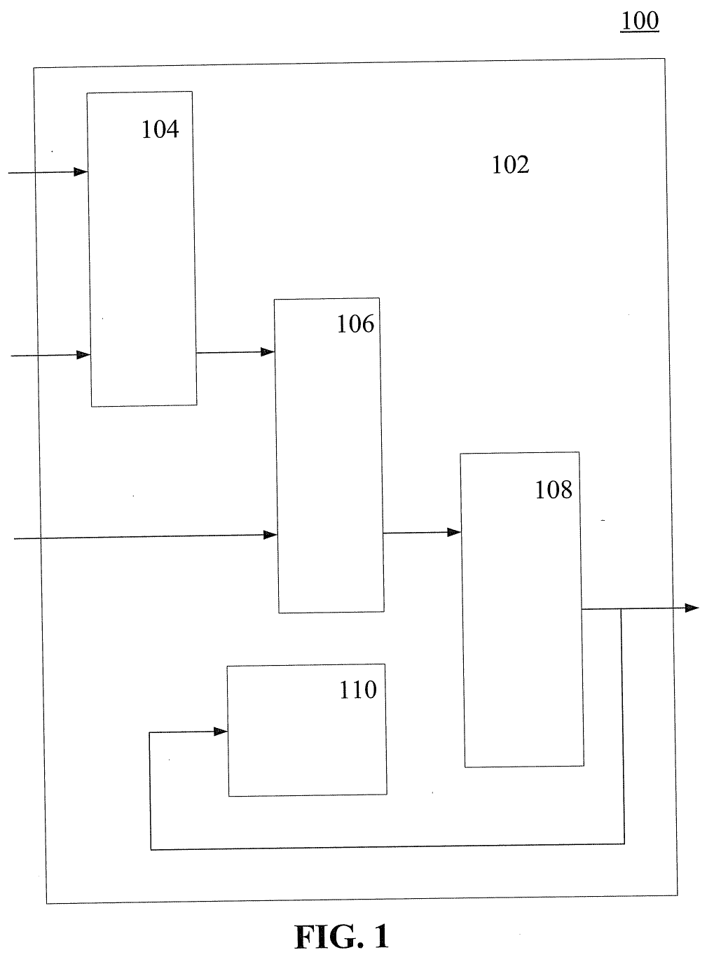

Referring initially to FIG. 1, a system 100 for modeling the wellness of an individual is schematically illustrated. The system 100 illustratively includes one or more electronic data processing elements 102. Additionally, the system includes a population profile integrator 104, an individual health profile comparator 106, and a wellness modeler 108. Preferably, the population profile integrator 104, individual health profile comparator 106, and wellness modeler 108 comprise computer-executable code configured to execute on the one or more electronic data processing elements 102 for performing the various functions or procedures described herein. In an alternate embodiment, however, one or more of these elements can be implemented in dedicated hardwired circuitry. In still another embodiment, the population profile integrator 104, individual health profile comparator 106, and/or wellness modeler 108 can be implemented in a combination of dedicated circuitry and/or computer-executable code.

Operatively, the population profile integrator 104 is configured to integrate pre-determined health indicators with statistically-valid health data derived from a sample population. The individual health profile comparator 106 is configured to compare the integrated health indicators and health data to individual-specific health data. The wellness modeler 108 is configured to generate a wellness assessment of an individual based on the comparison of the integrated health indicators and health data to the individual-specific health data.

By aggregating and synthesizing information, the system 100 can be used so as to develop for a particular individual an individualized health wellness program. Thus, by integrating reliable and accepted measurements of an individual's health with statistically valid samples of trends identified within a target population and the person's own health status and characteristics, the system 100 can be utilized for profiling an individual's wellness and for simulating certain changes, such as those of behavior and/or lifestyle, that would lead to either an improvement or deterioration in the overall wellness of the individual.

Accordingly, one aspect of the system 100 is the capability to simulate changes in the individual's lifestyle and/or behavior and project outcomes that stem from such changes. Thus, optionally, the system 100 can further comprise a re-calculation and projection tool 110. The re-calculation and projection tool 110 can be configured to project probabilistic health results based on simulated variations of values of the individual-specific health data.

According to one embodiment, the wellness modeler 108 is further configured to generated an optimized wellness program for the individual based upon the simulated variations in the values of the individual-specific health data. The optimized wellness program can comprise recommended behavioral actions corresponding to the simulated variations in the values of the individual-specific health data.

The system 100 can utilize, for example, key health indicators identified by the healthcare industry. Such indicators typically include an individual's weight, blood pressure, cholesterol levels, body mass index, and/or factors, behavioral and/or environmental, such as whether or not the individual is a cigarette smoker, suffers stress, and/or is exposed to various kinds of pollutants.

Determinations of wellness can be based upon valid data ranges. Actual trends and ranges for wellness measurements can vary by age, gender, genetic type, and various other factors. Utilizing health-relevant data provides a more precise measurement of wellness. Accordingly, the population profile integrator 104 can be further configured to identify predetermined best-practice health measurements corresponding to each of the pre-determined health indicators prior and to integrate the best-practice health measurements with the statistically-valid health data derived from a sample population. The individual health profile comparator 106 can be further configured to compare numbers, value ranges, and trends to current individual-specific health data to determine a numerical relationship between individual health measurements and population-based health measurements. Thus actual numbers can be mapped to determine specific measurements of health. Target numbers can be factored into the wellness modeler 108 so as to simulate or mode projected outcomes from certain health habit changes made by an individual.

The population profile integrator 104, thus, can combine best-practice health measurements with current and statistically relevant numbers or ranges from a desired population base. By comparing industry-established and/or demographically-relevant numbers, ranges, and trends to an individual's actual health data, the health profile comparator 106 can position a number of health factors relative to a larger, relevant population. The wellness modeler 108, given an individual's health factors, can utilize desired levels of identified health variables to model the overall wellness impact of certain actions and/or behaviors. From this, an optimized wellness program can be structured. In particular, the optionally-included re-calculation and projection tool 110 can alter values of certain health variables so as to project possible health implications and outcomes. This allows an individual to optimize and proactively develop a customized health plan.

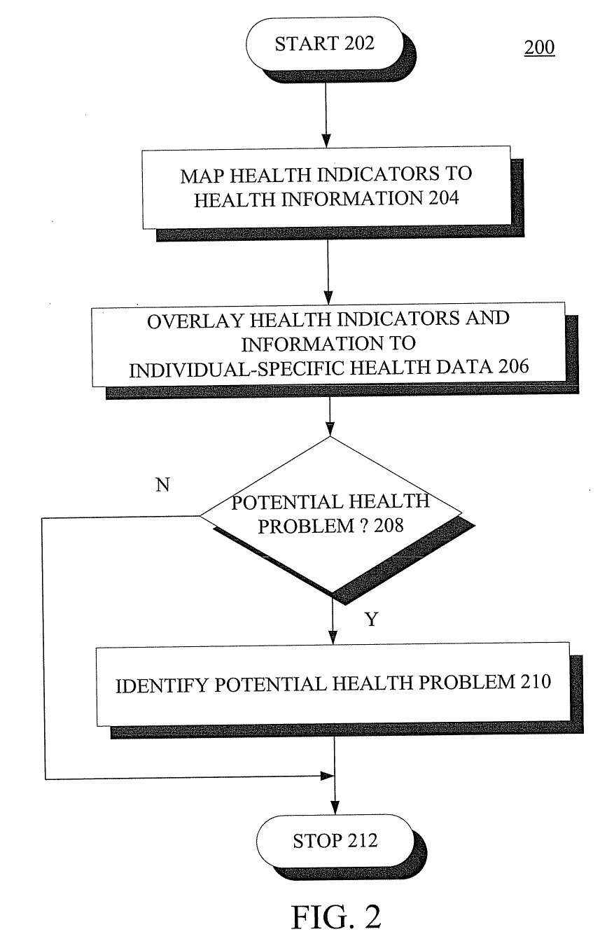

Referring now to FIG. 2, certain method aspects of the invention are illustrated. FIG. 2 is a flowchart of exemplary steps in a method 200 for developing an individualized wellness program, according yet another embodiment of the invention. The method 200, after the start at step 202, illustratively includes mapping or integrating a plurality of pre-determined health indicators to a database of health-related information at step 204. The method continues at step 206 by overlaying the pre-determined health indicators and health-related information to current individual-specific health data. Based on the overlaying step 206, it can be determined at step 208, according to the method 200, whether at least one individual health factor of an individual indicates a potential health problem. If at least one health factor indicates a potential health problem, the potential health problem can be identified at step 210. The method illustratively concludes at step 212.

According to another embodiment, the method 200 further includes generating the projected best course of individual action for maintaining or enhancing health of the individual. The step of generating a projected best course can comprise identifying and targeting at least one statistically valid behavioral action of the individual.

According to still another embodiment, the method 200 can further include determining trends in the health-related information. Yet another embodiment of the method 200 includes determining a range of values for at least one of the pre-determined health indicators. Moreover, the step of determining whether at least one individual health factor of an individual indicates a potential health problem can comprise determining whether the at least one individual health factor falls outside the range of values for a corresponding pre-determined health indicator. Additionally, the method 200 can further include projecting a change in health of the individual based on a pre-determined behavioral action corresponding to the at least individual health factor.

The invention, as already noted, can be realized in hardware, software, or a combination of hardware and software. The invention can be realized in a centralized fashion in one computer system, or in a distributed fashion where different elements are spread across several interconnected computer systems. Any kind of computer system or other apparatus adapted for carrying out the methods described herein is suited. A typical combination of hardware and software can be a general purpose computer system with a computer program that, when being loaded and executed, controls the computer system such that it carries out the methods described herein.

The invention, as also already noted, can be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

The foregoing description of preferred embodiments of the invention have been presented for the purposes of illustration. The description is not intended to limit the invention to the precise forms disclosed. Indeed, modifications and variations will be readily apparent from the foregoing description. Accordingly, it is intended that the scope of the invention not be limited by the detailed description provided herein.

We claim:

1. A computer-implemented method for developing an individualized wellness program, the method comprising:
mapping a plurality of pre-determined health indicators to a database of health-related information;
overlaying the pre-determined health indicators and health-related information to current individual-specific health data; and
based on the overlaying step, determining whether at least one individual health factor of an individual indicates a potential health problem and, if at least one health factor indicates a potential health problem, identifying the potential health problem.

2. The method of claim 1, further comprising generating the projected best course of individual action for maintaining or enhancing health of the individual.

3. The method of claim 2, wherein the step of generating a projected best course comprises identifying and targeting at least one statistically valid behavioral action of the individual.

4. The method of claim 1, further comprising determining trends in the health-related information.

5. The method of claim 1, further comprising determining a range of values for at least one of the pre-determined health indicators.

6. The method of claim 5, wherein the step of determining whether at least one individual health factor of an individual indicates a potential health problem comprises determining whether the at least one individual health factor falls outside the range of values for a corresponding pre-determined health indicator.

7. The method of claim 6, further comprising projecting a change in health of the individual based on a pre-determined behavioral action corresponding to the at least individual health factor.

8. A system for modeling the wellness of an individual, the system comprising:
at least one electronic data processing element;
a population profile integrator configured to execute on the electronic data processing element for integrating pre-determined health indicators with statistically-valid health data derived from a sample population;
an individual health profile comparator configured to execute on the electronic data processing element for comparing the integrated health indicators and health data to individual-specific health data; and
a wellness modeler configured to execute on the electronic data processing element for generating a wellness assessment of an individual based on the comparison of the integrated health indicators and health data to the individual-specific health data.

9. The system of claim 8, further comprising a re-calculation and projection tool configured to execute on the electronic data processing element for projecting probabilistic health results based on simulated variations of values of the individual-specific health data.

10. The system of claim 9, wherein the wellness modeler is further configured to generated an optimized wellness program for the individual based upon the simulated variations in the values of the individual-specific health data.

11. The system of claim 10, wherein the optimized wellness program comprises recommended behavioral actions corresponding to the simulated variations in the values of the individual-specific health data.

12. The system of claim 8, wherein the population profile integrator is further configured to identify predetermined best-practice health measurements corresponding to each of the pre-determined health indicators and to integrate the best-practice health measurements with the statistically-valid health data derived from a sample population.

13. The system of claim 8, wherein the individual health profile comparator is further configured to compare numbers, value ranges, and trends to current individual-specific health data to determine a numerical relationship between individual health measurements and population-based health measurements.

14. A computer-readable storage medium having stored therein a computer-readable instructions that when loaded in and executed by a computer causes the computer to:
map a plurality of pre-determined health indicators to a database of health-related information;
overlay the pre-determined health indicators and health-related information to current individual-specific health data; and
based on the overlaying step, determine whether at least one individual health factor of an individual indicates a potential health problem and, if at least one health factor indicates a potential health problem, identifying the potential health problem.

15. The computer-readable storage medium of claim 14, further comprising computer-readable instructions for causing the computer to generate a projected best course of individual action for maintaining or enhancing health of the individual.

16. The computer-readable storage medium of claim 15, wherein generating the projected best course comprises identifying and targeting at least one statistically valid behavioral action of the individual.

17. The computer-readable storage medium of claim 14, further comprising computer-readable instructions for causing the computer to determine trends in the health-related information.

18. The computer-readable storage medium of claim 14, further comprising computer-readable instructions for causing the computer to determine a range of values for at least one of the pre-determined health indicators.

19. The computer-readable storage medium of claim 18, wherein the step of determining whether at least one individual health factor of an individual indicates a potential health problem comprises determining whether the at least one individual health factor falls outside the range of values for a corresponding pre-determined health indicator.

20. The computer-readable storage medium of claim 19, further comprising computer-readable instructions for causing the computer to project a change in health of the individual based on a pre-determined behavioral action corresponding to the at least individual health factor.
